(19) United States
(12) Patent Application Publication
(10) Pub. No.: US 2004/0106114 A1
(43) Pub. Date: Jun. 3, 2004

(54) Simplified detection process for colored bead random microarrays

(75) Inventor: Thomas E. Kocher, Rochester, NY (US)

Correspondence Address: Thomas H. Close, Patent Legal Staff, Eastman Kodak Company, 343 State Street, Rochester, NY, 14650-2201, US

(73) Assignee: Eastman Kodak Company

(21) Appl. No.: 10/307,664

(22) Filed: Dec. 2, 2002

(51) Int. Cl. C12Q 1/68; G06K 9/00; C12M 1/34; B05D 3/00

(52) U.S. Cl. 435/6; 435/287.1; 427/2.11; 382/128

(57) ABSTRACT

A method of manufacturing a colored microsphere molecular biological/chemical random microarray system, comprising: randomly dispersing on a support a composition including a first set of microspheres modified with a biological/chemical probe and containing an optical bar code generated from at least one colorant associated with the microspheres to produce a biologically/chemically active region on the support; scanning the biologically/chemically active region with a high-resolution color scanner to produce a color map of the locations of the randomly dispersed first set of microspheres; creating a digital file of the color map; and linking the digital file of the color map with the support.

### FIELD OF THE INVENTION

This invention relates in general to molecular biological systems and more particularly to a means to simplify the detection process for colored bead random microarrays.

### BACKGROUND OF THE INVENTION

Ever since it was invented in the early 1990s (Science, 251, 767-773, 1991), high-density arrays formed by spatially addressable synthesis of bioactive probes on a 2-dimensional solid support has greatly enhanced and simplified the process of biological research and development. The key to current microarray technology is deposition of a bioactive agent at a single spot on a microchip in a “spatially addressable” manner.

Current technologies have used various approaches to fabricate microarrays. For example, U.S. Pat. No. 5,412,087, inv. McGall et al., issued on May 2, 1995 and U.S. Pat. No. 5,489,678, inv. Fodor et al., issued Feb. 6, 1996, demonstrate the use of a photolithographic process for making peptide and DNA microarrays. The patent teaches the use of photolabile protecting groups to prepare peptide and DNA microarrays through successive cycles of deprotecting a defined spot on a 1 cm×1 cm chip by photolithography, then flooding the entire surface with an activated amino acid or DNA base. Repetition of this process allows construction of a peptide or DNA microarray with thousands of arbitrarily different peptides or oligonucleotide sequences at different spots on the array. This method is expensive. An ink jet approach is being used by others (e.g., Papen et al., U.S. Pat. No. 6,079,283, issued Jun. 27, 2000, U.S. Pat. No. 6,083,762; issued, Jul. 4, 2000 and U.S. Pat. No. 6,094,966, issued Aug. 1, 2002) to fabricate spatially addressable arrays, but this technique also suffers from high manufacturing cost in addition to the relatively large spot size of 40 to 100 μm. Because the number of bioactive probes to be placed on a single chip usually runs anywhere from 1000 to 100000 probes, the spatial addressing method is intrinsically expensive regardless how the chip is manufactured.

An alternative approach to the spatially addressable method is the concept of using fluorescent dye-incorporated polymeric beads to produce biological multiplexed arrays. U.S. Pat. No. 5,981,180, inv. Chandler et al., issued Nov. 9, 1999 discloses a method of using color coded beads in conjunction with flow cytometry to perform multiplexed biological assay. Microspheres conjugated with DNA or monoclonal antibody probes on their surfaces were dyed internally with various ratios of two distinct fluorescence dyes. Hundreds of “spectrally addressed” microspheres were allowed to react with a biological sample and the “liquid array” was analyzed by passing a single microsphere through a flow cytometry cell to decode sample information. U.S. Pat. No. 6,023,540, inv. Walt et al., issued Feb. 8, 2000 discloses the use of fiber-optic bundles with pre-etched microwells at distal ends to assemble dye loaded microspheres. The surface of each spectrally addressed microsphere was attached with a unique bioactive agent and thousands of microspheres carrying different bioactive probes combined to form “beads array” on pre-etched microwells of fiber optical bundles. More recently, a novel optically encoded microsphere approach was accomplished by using different sized zinc sulfide-capped cadmium selenide nanocrystals incorporated into microspheres (Nature Biotech. 19, 631-635, (2001)). Given the narrow band width demonstrated by these nanocrystals, this approach significantly expands the spectral barcoding capacity in microspheres.

Even though the “spectrally addressed microsphere” approach does provide an advantage in terms of its simplicity over the old fashioned “spatially addressable” approach in microarray making, there are still needs in the art to make the manufacture of biological microarrays less difficult and less expensive.

### SUMMARY OF THE INVENTION

According to the present invention, there is provided a solution to the problems of the prior art.

According to another feature of the present invention, there is provided a method of manufacturing a colored microsphere molecular biological/chemical random microarray system, comprising:

randomly dispersing on a support a composition including a first set of microspheres modified with a biological/chemical probe and containing an optical bar code generated from at least on colorant associated with said microspheres to produce a biologically/chemically active scanning region on said support;

scanning said biologically/chemically active region with a high-resolution color scanner to produce a color map of the locations of said randomly dispersed first set of microspheres;

creating a digital file of said color map; and

linking said digital file of said color map with support.

### ADVANTAGEOUS EFFECT OF THE INVENTION

The invention has the following advantages.

1. Reduces the unit manufacturing cost of the random array reader.

2. Increases the spatial resolution in the case where color CCD detection is used.

### BRIEF DESCRIPTION OF THE DRAWINGS

FIG. 1 is a block diagram of the manufacturing protocol according to an embodiment of the present invention.

FIG. 2 is a block diagram of the readout protocol according to another embodiment of the present invention.

FIG. 3 is a diagrammatic view showing a color bead random array support with fiducial marks, magnetic strip, and barcode.

### DETAILED DESCRIPTION OF THE INVENTION

In general, according to the present invention, there is provided a color bead random array, wherein protein antibodies attached to beads are identified by a specific color. That is, in the manufacturing process, colored microspheres (beads) have attached protein antibodies such that a unique and discernable color can be associated with a unique and discernable antibody. In the application of random bead arrays for biomolecular detection, fluorescently or chemiluminescently labeled biological target sample interacts with a biological probe (e.g. protein antibody). Specific binding occurs between the target sample and the probe. In the readout process, this specific binding is detected. In addition, the binding probe needs to be uniquely identified to determine uniquely the nature of the sample that interacted with it. With color bead random array technology, the specific probe is identified through its unique color. Typically this color is identified by using a color CCD detector or by using a monochrome detector and using an illuminant of wavelength that can be varied. In either case, there is significant cost associated with the readout/detection means to identify this color information. In this invention, the color beads are identified during the manufacturing process and these data are linked to the array through a barcode or magnetic encoding means via a digital file. The manufacturing line will have a high-resolution color scanner to map the positions and color of the beads and store this as an electronic file. The random array could require the addition of imaging fiducials or simple unique identifiers to better discern bead location. The readout device (reader) would not require a means to identify color, only a means to identify luminance intensity associated with the fluorescent or chemiluminescent label at a specific location.

This invention is reduces the unit manufacturing cost (UMC) of the random array reader by eliminating the need for a multiple wavelength illuminant source, a color scanner/CCD array and increases the spatial resolution in the case where color CCD detection is used because the color mask required by the CCD is not required in the monochrome detection case.

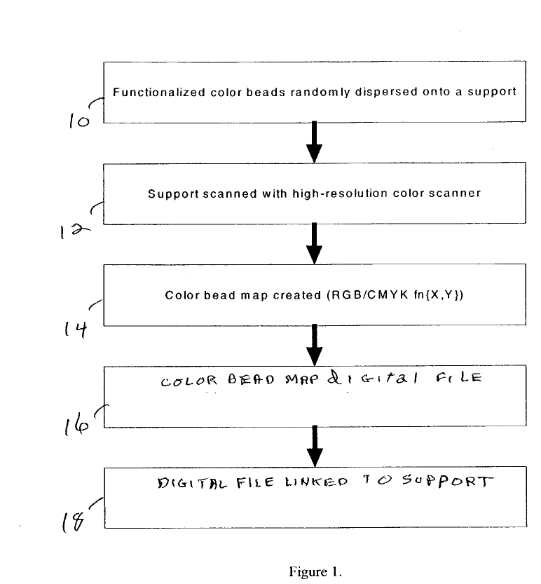

Referring now to FIG. 1 there is shown a block diagram of a method according to the invention. As shown, a method of manufacturing a color microsphere molecular biological/chemical random microarray includes the following processes. (1) Randomly dispersed on a support is a composition (a) including a first set of microspheres modified with a biological/chemical probe and (b) containing an optical bar code generated from at least one colorant associated with the microspheres to produce a biologically/chemically active region (box 10). (2) The biologically/chemically active region is scanned with a high-resolution color scanner (box 12) to produce a color map of the locations of the randomly dispersed first set of microspheres (box 14). (3) A digital file of the color map of microspheres is created (box 16). (4) The digital file of the color map of microspheres is linked to the support (box 18).

Preferably, the composition dispersed on the support (a) includes a plurality of sets of microspheres, each set being modified with a unique biological/chemical probe and (b) contains a unique optical bar code generated from at least one colorant associated with the plurality of sets of microspheres; and scanning the active region produces a color map of the locations of the randomly dispersed plurality of sets of microspheres.

Referring to FIG. 3, there is shown a diagrammatic view of a microarray system 30 including a biologically/chemically active region 32 on support 34. Region 32 has a randomly dispersed composition including a plurality of sets of microspheres 36, each set being modified with a unique biological/chemical probe and containing a unique optical bar code generated from at least one colorant associated with the plurality of sets of microspheres. A digital map of a color map of the randomly dispersed microspheres is stored in one or both of magnetic strip 38 and optical bar code 40. The digital file of the color map can also be stored in an RFID region on support 34.

Fiducial marks 42 are provided as a reference to locate system 30 in a utilization system. The fiducial marks 42 can be, but aren't limited to, identifiable opaque mark(s) on the array, a physical notch(s) in the array support, or an identifiable mark in the support coating, such as a region of higher or lower reflectivity. This fiducial mark could also be part of the barcode or magnetic strip or human readable identifiers on the support.

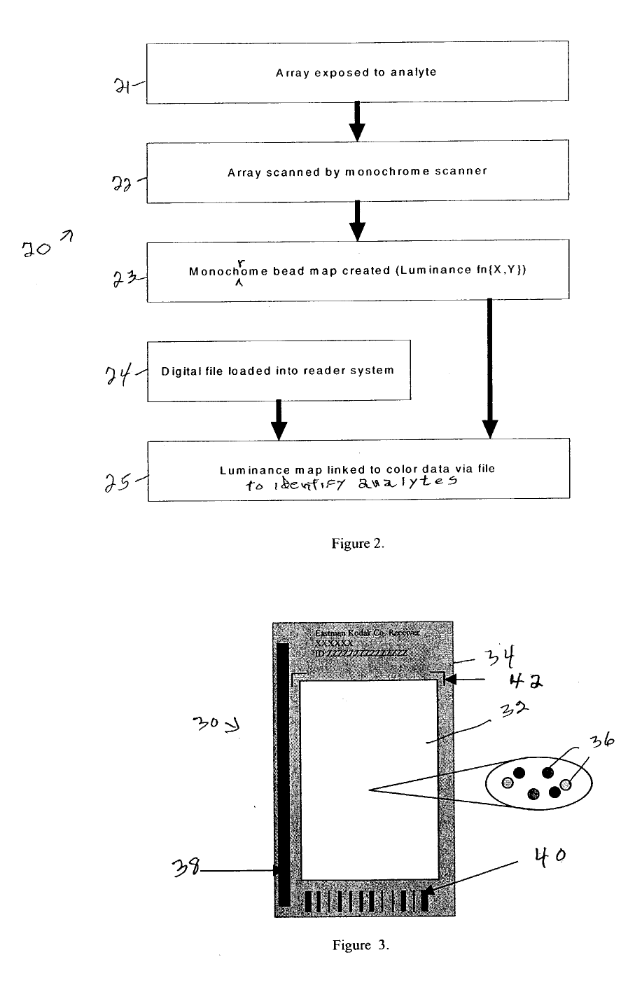

Referring now to FIG. 2, a method of utilizing the microarray system of the present invention is shown. Method 20 includes the following processes. (1) Microarray system 30 is exposed to an analyte (box 21). (2) Exposed microarray system 20 is scanned by a monochrome scanner (box 22) to produce a monochrome bead (microsphere) map [luminance fn (X,Y)] (box 23). (3) The digital file of the color bead map linked to system 30 is read and loaded into a reader system (box 24). (4) The luminance bead map is linked to the color data of the color bead map to identify the exposed analytes.

The reader system can be a digital computer or microcomputer based system for digitally processing the digital color bead map and monochrome bead map to identify the exposed analytes.

The invention has been described in detail with particular reference to certain preferred embodiments thereof, but it will be understood that variations and modifications can be effected within the spirit and scope of the invention.

### Parts List

10 functionalized color beads randomly dispersed onto a support

12 support scanned with high-resolution color scanner

14 color bead map created (RGB/CMYK fn {X,Y})

16 color bead map digital file

18 digital file linked to support

21 array exposed to analyte

22 array scanned by monochrome scanner

23 monochrome bead map created (luminance fn {X,Y})

24 digital file loaded into reader system

25 luminance map linked to color data via file to identify analytes

30 microarray system

32 region

34 biological/chemical active region

36 microarrays

38 magnetic strips

40 optical bar code

42 fiducial marks

What is claimed is:

1. A method of manufacturing a colored microsphere molecular biological/chemical random microarray system, comprising:
randomly dispersing on a support a composition including a first set of microspheres modified with a biological/chemical probe and containing an optical bar code generated from at least one colorant associated with said microspheres to produce a biologically/chemically active region on said support;
scanning said biologically/chemically active region with a high-resolution color scanner to produce a color map of the locations of said randomly dispersed first set of microspheres;
creating a digital file of said color map; and
linking said digital file of said color map with said support.

2. The method of claim 1 wherein said composition dispersed on said support (a) includes a plurality of sets of microspheres, each set being modified with a unique biological/chemical probe and (b) contain a unique optical bar code generated from at least one colorant associated with said plurality of sets of microspheres; and
wherein said scanning produces a color map of the locations of said randomly dispersed plurality of sets of microspheres.

3. The method of claim 1 wherein said linking includes storing said digital file magnetically, optically or electronically on said support.

4. The method of claim 1 wherein said linking includes storing said digital file in a magnetic region on said support.

5. The method of claim 1 wherein said linking includes storing said digital file in an optical bar code region on said support.

6. The method of claim 1 wherein said linking includes storing said digital file in a radio frequency identification (RFID) region on said support.

7. The method of claim 1 wherein said support has fiducial marks useful for reference purposes.

8. A colored microsphere molecular biological/chemical random microarray system comprising:
a support;
a biologically/chemically active region on said support, said region having a randomly dispersed composition (a) including a first set of microspheres modified with a biological/chemical probe and (b) containing an optical bar code generated from at least one colorant associated with the microspheres; and
a data region linked to said support outside of said active region having a stored digital file of a color map of the locations of said randomly dispersed first set of microspheres.

9. The system of claim 8 wherein said active region has a randomly dispersed composition which (a) includes a plurality of sets of microspheres, each set being modified with a unique biological/chemical probe, and (b) contains a unique optical bar code generated from at least one colorant associated with said plurality of sets of microspheres.

10. The system of claim 1 wherein said digital file is stored magnetically, optically or electronically in said data region on said support.

11. The system of claim 1 wherein said digital file is stored in a magnetic data region on said support.

12. The system of claim 1 wherein said digital file is stored in an optical bar code data region on said support.

13. The system of claim 1 wherein said digital file is stored in an RFID region on said support.

14. The system of claim 1 wherein said support has fiducial marks useful for reference purposes.

15. A method of identifying at least one biological/chemical analyte in a sample containing said analyte comprising:
providing a colored microsphere molecular biological/chemical random microarray system including (I) a support having a biologically/chemically active region with a randomly dispersed composition (a) including a first set of microspheres modified with a biological/chemical probe and (b) containing an optical bar code generated from at least one colorant associated with the microspheres, and (II) a data region linked to said support outside of said active region having a stored digital file of a color map of the locations of said randomly dispersed first set of microspheres;
exposing said active region of said system to an analyte;
scanning exposed said active region by a monochrome microsphere map;
reading with a digital reader said data region to load said digital file of said color microsphere map into said digital reader;
processing said monochrome microsphere map and said color microsphere map to identify the identity of the exposed analyte(s).
